(19) United States
(12) Patent Application Publication
(10) Pub. No.: US 2011/0009660 A1
(43) Pub. Date: Jan. 13, 2011

(54) Process for the removal of HF from HF containing organic carbonates

(75) Inventors: Harald Krueger, Bad Wimpfen (DE); Christoph Sommer, Neckarsulm (DE); Alain Lambert, Beauvechain (BE); Phillip Schwerdt, Mainz (DE); Jens Olschimke, Hannover (DE); Dirk Seffer, Neustadt (DE); Saskia Wedekind, Sarstedt (DE)

Correspondence Address: Solvay;c/o B. Ortego - IAM-NAFTA, 3333 Richmond Avenue, Houston, TX, 77098-3099, US

(73) Assignee: SOLVAY FLOUR GMBH, Hannover (DE)

(21) Appl. No.: 12/933,503

(22) PCT Filed: Mar. 26, 2009
(86) PCT No.: PCT/EP2009/053561

§ 371 Date: Sep. 20, 2010

(30) Foreign Application Priority Data
Mar. 27, 2008 (EP) 08153413.3
Mar. 28, 2008 (EP) 08153481.0

(51) Int. Cl. C07C 68/08

(52) U.S. Cl. 558/260

(57) ABSTRACT

Mixtures comprising organic carbonates, such as carbonates not substituted by fluorine, but especially fluorosubstituted organic carbonates, which are depleted in HF can be obtained from respective mixtures with a higher HF content by a step of stripping such mixture with an inert gas. For example, a reaction mixture comprising fluoroethylene carbonate and HF can be treated in this way. The mixture comprising organic carbonate which is depleted in HF can be distilled to obtain highly purified organic carbonate.

The present invention concerns a process for the removal of hydrogen fluoride (HF) from mixtures comprising HF and organic carbonates, especially fluorosubstituted organic carbonates.

Fluorosubstituted organic carbonates, for example, mono-, di- and trifluoroethylene carbonates, and fluorinated dimethyl carbonates, for example, fluoromethyl methyl carbonate, 1,1-difluoromethyl carbonate, 1,2-difluoromethyl methyl carbonate, and dimethyl carbonates with even higher degree of fluorination, for example, the trifluorinated and tetrafluorinated compounds, are especially suitable as solvents or solvent additives for lithium ion batteries.

Fluoroethylene carbonate, for example, can be prepared from the respective unsubstituted ethylene carbonate by the reaction of 1,3-dioxolane-2-one (ethylene carbonate/“EC”) with elemental fluorine. This is described for example in JP-A 2000-309583 where the reaction is performed with a melt of EC or its solution in anhydrous fluoride. Optionally, perfluorohexane can be present; here, a suspension of 1,3-dixolane-2-one is formed. According to U.S. patent application Ser. No. 2006-0036102, ethylene carbonate is dissolved in F1EC and then contacted with fluorine. According to U.S. Pat. No. 7,268,238, the reaction is performed in a column integrated into a reactor with Raschig rings to provide a suitable bubble size of the fluorine gas. Di- and trifluoroethylene carbonates can either be prepared from ethylene carbonate wherein a respective higher molar ratio of fluorine is introduced into the reaction. Alternatively, monofluorinated ethylene carbonate can be reacted with further fluorine. This is described in JP 2000-344763.

M. Kobayashi et al. disclose in J. Fluorine Chem. 120 (2003), pages 105 to 110 a process for the manufacture of fluoroethylene carbonate by direct fluorination of ethylene carbonate. In that process, the reactor and the lines are purged with nitrogen. HF is removed from the reaction mixture by washing it with water.

EP-A-0 557167 describes the manufacture of fluorinated functional compounds via a carbonate intermediate which is prepared by direct fluorination of organic carbonates. Volatilized HF is purged from the reactor.

Fluorosubstituted propylene carbonates, fluorosubstituted dimethylcarbonates, difluoropropylene carbonates and other fluorosubstituted carbonates can be prepared in a comparable manner.

During the reaction of fluorine with the organic carbonate, hydrogen fluoride is formed as co-product. It may additionally be present in the reaction mixture if it was used as solvent.

For isolation of the desired reaction product, HF has to be removed from the organic constituents. According to the documents cited above, this is achieved by aqueous workup or by distillation of the crude reaction mixture.

Object of the present invention is to provide a simple process for the removal of HF from its mixture with organic carbonate, be they fluorinated or not.

According to the present invention, mixtures with depleted hydrogen fluoride content are prepared from a mixture comprising organic carbonate, preferably fluorinated organic carbonate, and hydrogen fluoride by stripping HF from the mixture by passing inert gas through the mixture. Noble gases or their mixtures with nitrogen or carbon dioxide or its mixtures with nitrogen are also suitable as inert gas for stripping; air also might be suitable, but it is not preferred. Nitrogen is especially suitable as stripping gas.

The singular form “carbonate” is intended to include the plural form; thus, the term “mixture comprising organic carbonate” also denotes a mixture comprising two or more organic carbonates.

Preferably, the mixture of organic carbonate is not contacted or washed with water, neither before nor after the stripping.

According to one embodiment, the process is applied to separate HF from alkylene carbonates, for example, vinylene carbonate, ethylene carbonate, or propylene carbonate, or from dialkyl carbonates. Alkyl denotes preferably C1 to C4 alkyl. The alkyl groups can be the same or different. Especially preferably, they stand for methyl or ethyl.

Preferably, the mixtures to be treated are reaction mixtures resulting from fluorination reactions between nonfluorinated organic carbonates or fluorinated organic carbonates and fluorine to provide product with as higher degree of fluorination than the starting compound(s).

Preferably, the HF-containing reaction mixture results from a non-fluorinated organic carbonate starting material which is fluorinated with elemental fluorine to form a fluorosubstituted organic carbonate reaction product and HF. In this type of reaction, undiluted fluorine could be used. For safety reasons, usually fluorine/inert gas mixtures are applied, especially fluorine/nitrogen mixtures. Passing such a reactive gas mixture through the starting material is not considered as stripping in the context of the present invention. In the present invention, stripping is performed with inert gas which does not react with the constituents of the reaction mixture, especially, which does not further fluorinate unreacted starting material.

According to one embodiment, fluorine free dialkyl carbonates or alkylene carbonates are applied as starting material which is fluorinated and gives mixtures of HF and fluorinated carbonates from which HF is removed according to the process of the present invention. Concerning dialkyl carbonates, the alkyl groups can be the same or different and preferably denote C1 to C4 alkyl groups. They may be different and preferably denote methyl or ethyl, or they are, which is especially preferred, the same and denote methyl or ethyl. Concerning alkylene carbonates, the term “alkylene” denotes preferably a C2 to C6 alkylene group. A C2 alkylene group is preferably included in the ring, i.e. it represents the compound ethylene carbonate, or 1,3-dioxolane-2-one. If the alkylene group is C3 group, preferably two of the three carbon atoms are included in the ring, and thus, the preferred compound is 4-methyl-1,3-dioxolane-2-one. If the alkylene group is a C4 to C6 group, then the preferred compounds are those which form a 5-membered ring, with alkyl substituents at the 4-carbon atom or the 4-carbon atom and the 5-carbon atom. Especially preferred are dimethyl carbonate, methyl ethyl carbonate, diethyl carbonate, 4,5-dimethyl-1,3-dioxolane-2-one, 4-ethyl-1,3-dioxolane-2-one, 4-methyl-5-ethyl-1,3-dioxolane-2-one, 4-n-propyl-1,3-dioxolane-2-one, 4-i-propyl-1,3-dioxolane-2-one, 4-vinyl-1,3-dioxolan-2-one, 1,3-dioxo1-2-one, 4-ethyl-5-methyl-1,3-dioxolan-2-one, and 4,5-ethyl-1,3-1,3-dioxolane-2-one.

In another embodiment, a starting material is applied which consists of or comprises dialkyl or alkylene carbonates which are already substituted by at least one fluoro atom, which are reacted to give higher fluorinated material in admixture with HF from which mixtures HF is removed by the process of the present invention. For example, fluoroethylene carbonate can be applied as starting material to be fluorinated to form difluoroethylene carbonate or even higher fluorinated compounds. It is also possible to apply a mixture of non-fluorinated organic carbonates and fluorosubstituted organic carbonates. For example, a mixture of fluoroethylene carbonate and ethylene carbonate may be applied as starting material. Here it is possible to fluorinate ethylene carbonate to form fluoroethylene carbonate, or, when a higher amount of fluorine is applied, even to form difluoroethylene carbonate. Of course, one also can apply starting material which contains higher fluorinated compounds besides compounds with a lower degree of fluorination, or which are not fluorinated at all. For example, a mixture which comprises ethylene carbonate, fluoroethylene carbonate and difluoroethylene carbonate can be reacted with elemental fluorine to obtain a mixture with an increased content of fluoroethylene carbonate.

Thus, the preferred reaction mixtures from which HF is removed by stripping according to the present invention comprise, according to one preferred embodiment, nonfluorinated starting material, dialkyl carbonate or alkylene carbonate substituted by one or more fluorine atoms, and HF. According to another embodiment, the reaction mixtures comprise fluorosubstituted dialkyl or alkylene carbonates with lower and higher degree of fluorination and HF.

Mixtures which contain HF in a broad range can be treated according to the present invention. In the most preferred embodiments where the reaction mixture to be treated originates from the preparation of fluorosubstituted ethylene carbonates or fluorosubstituted dialkyl carbonates, one molecule of HF is formed per hydrogen atom which is substituted by fluorine. Usually, in such reaction mixtures, the content of HF is equal to or lower than 10% by weight. But mixtures which comprise higher amounts of HF can be treated, too.

The content of HF in the mixtures after treatment is preferably equal to or lower than 2% by weight of the reaction mixture. Preferably, it is equal to or lower than 1% by weight. Still more preferably, it is equal to or lower than 0.5% by weight. Especially preferably, it is equal to or lower than 0.1% by weight.

In a most simple manner, stripping could be performed in a vessel containing reaction mixture by blowing inert gas through the reaction mixture. This can be done batch wise or continuously.

It is preferred to perform stripping in a way which provides for a sufficient contact area between reaction mixture and gas. For example, reaction mixture could be sprayed into a stream of inert gas, or stripping gas and liquid to be treated can be contacted in a bubble tray column. A very preferred method is performed in a stripping column. In a stripping column, internals or packings are installed with a high specific area per m³ of the equipment to provide a high contact surface between gas and liquid. Suitable packings are, for example, Raschig rings. The stripping column is usually a cylindrical tube positioned vertically. The inert gas is introduced at the bottom of the stripping column below the packings, the reaction mixture is fed at the top. Inert gas comprising HF leaves the column via a separate line at the top.

The efficiency of the removal of HF from the HF-containing carbonate is higher at higher temperatures. If the contact is performed in a vessel, heat can be supplied in a known manner, for example, by heating the walls of the vessel. Optionally, the inert gas and/or the liquid to be treated can be heated.

If the reaction is performed in a stripping column with internals or packings, it is preferred to heat inert gas, liquid to be treated or both to improve the efficiency of the stripping process.

Thus, the inert gas, especially nitrogen, advantageously is heated before introducing it into the reaction mixture. The temperature to which it is heated is preferably equal to or higher than 60° C. ; more preferably, it is equal to or higher than 75° C. Very preferably, it is equal to or higher than 100° C. The temperature can still be higher, for example, equal to or higher than 120° C. Preferably, it is equal to or lower than 150° C. Depending on the heat resistance and the resistance of corrosion of the vessel, column, pipes, fittings etc used, the temperature can be higher than 150° C.

The reaction mixture preferably is also heated before a continuous stripping process is performed. If a vessel is used to perform a batch wise process, the reaction mixture can be heated before and/or during the stripping process. Preferably, it is heated to a temperature equal to or greater than 60° C. Preferably, it is heated to a temperature equal to or lower than 120° C.

It is very advantageous to perform the stripping step at ambient pressure. If desired, a slight vacuum can be applied. For example, the pressure can be reduced to 0.5 bar or even 0.2 bar. The temperature should not be so high that organic compounds would be carried out of it with the flow of inert gas.

In a batch wise process, stripping is performed until the desired maximal amount of HF is present.

In a continuous process in a stripping column, the height of the column is selected such that, for a given HF concentration, flow rate of inert gas and reaction mixture, the desired residual HF concentration is reached.

The reaction mixture leaving the stripping step can then be fed to one or more distillation columns to isolate pure product.

In a preferred embodiment, purified carbonate is produced by a subsequent step of at least one distillation. Thus, another object of the present invention is a process for the manufacture of purified organic carbonate from its mixture with HF, comprising at least one step of stripping the mixture of organic carbonate and HF by stripping HF from the mixture by passing an inert gas through the reaction mixture to obtain an intermediate product depleted in HF, and at least one distillation step wherein the intermediate product depleted in HF is distilled to obtain the purified organic carbonate. It is preferred that mixture of organic carbonate and HF is not contacted or washed with water before or after the stripping step or stripping steps, and also not before or after any distillation step.

The stripping process to remove HF has several advantages. A great advantage is that it obviates an aqueous workup. It may reduce the number of distillation steps needed to provide pure product. Thus, it may reduce thermal impact on the product, especially, if a continuously performed stripping process is performed in a stripping column.

It must be considered to be very surprising that stripping can effectively remove HF from the carbonates because it was found that HF has an extremely low activity coefficient in organic carbonates. In monofluoroethylene carbonate, for example, the activity coefficient y of HF was determined to be 0.08.

The following examples are intended to explain the stripping process in further detail without intending to limit it.

### Example 1

Batch stripping of HF from a reaction mixture comprising monofluoroethylene carbonate, under heating to 65° C.

Origin of the reaction mixture: Ethylene carbonate dissolved in monofluoroethylene carbonate as described in US-A 2006-0036102 was contacted with a fluorine/nitrogen mixture comprising 16% by weight of fluorine. The resulting reaction mixture of comprised about 7.1% by weight of HF. The remainder was mostly fluoroethylene carbonate and unreacted ethylene carbonate.

About 500 kg of the reaction mixture was filled into a vessel which could be heated via the walls. The vessel had an inlet to introduce nitrogen gas below the surface of the liquid reaction mixture and an outlet for the resulting HF/nitrogen gas mixture. The reaction mixture was heated to 65° C., and 10 kg/h of nitrogen (which was not heated before introduction into the reaction mixture) was passed through the mixture. The content of HF in the treated reaction mixture was analyzed regularly. Analysis gave the data presented in table 1:

TABLE 1

| Time [h] | Residual content of HF [g/kg] |
|---|---|
| 0 | 71 |
| 8 | 65 |
| 16 | 44 |
| 24 | 41 |
| 32 | 36 |
| 40 | 22 |
| 48 | 18 |
| 56 | 19 |
| 64 | 12 |
| 72 | 12 |
| 80 | 13 |
| 87.5 | 9.3 |
| 91.5 | 7.4 |
| 95.5 | 6.7 |
| 99.5 | 5.8 |
| 103.83 | 4.5 |
| 106.5 | 3.7 |

### Example 2

Batch stripping of HF from a reaction mixture comprising monofluoroethylene carbonate, under heating to 80° C.

Example 1 was repeated with a reaction mixture which initially contained 5.7% by weight of HF. This time, the temperature of the reaction mixture was kept at 80° C.

The analysis data of HF in the reaction at certain time intervals are given in table 2:

TABLE 2

| Time [h] | Residual content of HF [g/kg] |
|---|---|
| 0 | 57 |
| 4.25 | 36 |
| 8.25 | 32 |
| 12.25 | 26 |
| 16.25 | 21 |
| 20.25 | 17 |
| 24.25 | 13 |
| 28.25 | 10 |
| 32.25 | 9.8 |
| 36.25 | 8.6 |
| 40.25 | 7 |
| 44.25 | 5.9 |
| 48.25 | 5.6 |

The results in tables 1 and 2 show that the HF content can effectively reduced by stripping in spite of the very low activity coefficient of HF in monofluoroethylene carbonate. Further, the results show a low residual amount of HF is achieved in a much shorter time when stripping is performed at a higher temperature. Consequently, the nitrogen consumption is considerably reduced.

It has to be noted that the residual content of HF could have been further reduced if stripping would have continued. The achieved level of 3.7 and 5.6 g/kg in the mixture is by no means the final concentration.

### Example 3

Continuously performed stripping process

In this example, stripping is performed in a packed stripping column with 12 theoretical stages. Nitrogen gas is introduced into the column at the bottom below the packing, the liquid reaction mixture is introduced at the top of the column. Pressure is about 1.1 bars (abs.), the reaction mixture to be treated is heated to 90° C. before it is fed into the column, and nitrogen is heated to 120° C. before introduction into the column. The total flow of the reaction mixture is set to 65 kg/h, the flow of nitrogen to 112 kg/h.

The content of the reaction mixture before and after stripping is given in table 3. Used abbreviations:

EC=Ethylene carbonate

F1EC=monofluoroethylene carbonate

CIS-F2EC=cis-4,5-difluoro-dioxolane-2-one

TR-F2EC=trans-4,5-difluoro-dioxolane-2-one

4,4-F2EC=4,4-difluoro-dioxolane-2-one

HF=hydrogen fluoride

N2=nitrogen

| | Content in the reaction mixture | |
|---|---|---|
| Compound | Before stripping | After stripping |
| EC | 0,279 | 0.321 |
| F1EC | 0.509 | 0.578 |
| CIS-F2EC | 0.034 | 0.037 |
| 4,4-F2EC | 0.022 | 0.016 |
| TR-F2EC | 0.079 | 0.047 |
| HF | 0.077 | 6.51E-06 |
| N2 | 0 | 2.63E-6 |

The terms “6.51E-06” and “2.63E-06” denote 6.51 ppm and 2.63 ppm, respectively, demonstrating the excellent performance of a stripping column for HF removal from fluorinated organic carbonates.

It further has to be noted that the HF content will be further reduced during subsequent purification steps for isolating pure F1EC.

### Example 4

Stripping a reaction mixture from difluoroethylene carbonate production

Difluoroethylene carbonate is prepared by reaction of ethylene carbonate and a fluorine/nitrogen mixture comprising 16% by weight of fluorine. The fluorination is performed until a reaction mixture is obtained which comprises about 7% by weight HF and about 50% by weight of difluoroethylene carbonates (cis- and trans-difluoroethylene carbonate and 4,4-difluoroethylene carbonate). It further contains unreacted ethylene carbonate, monofluoroethylene carbonate and trifluoroethylene carbonate.

The reaction mixture is transferred to a vessel, heated to about 70° C. by means of heating elements arranged in the wall of the vessel, and nitrogen is passed through the liquid. Nitrogen is blown through the liquid until the HF content is reduced to 0.5% by weight.

The HF-depleted reaction mixture can be further treated to remove residual HF, e.g. by contacting it with suitable adsorbents or absorbents, e.g. silica. Then, the difluoroethylene carbonates can be isolated and separated from each other by subsequent distillation.

1. A process for the preparation of a mixture with depleted hydrogen fluoride content from a mixture containing an organic carbonate and hydrogen fluoride, said process comprising stripping HF from the mixture by passing an inert gas through the mixture containing said organic carbonate and hydrogen fluoride.

2. The process according to claim 1, wherein the inert gas used as stripping gas is selected from the group consisting of nitrogen, noble gases, carbon dioxide, and mixtures thereof.

3. The process according to claim 1, wherein the mixture which is stripped is a mixture comprising a fluorosubstituted organic carbonate and hydrogen fluoride.

4. The process according to claim 2, wherein the mixture which is stripped is a mixture comprising HF and an organic carbonate selected from the group consisting of monofluoroethylene carbonate, difluoroethylene carbonate, trifluoroethylene carbonate, and combinations thereof.

5. The process according to claim 1, wherein said stripping is performed batchwise.

6. The process according to claim 4, wherein the mixture to be stripped is heated before being fed into a vessel in which stripping is performed and/or heated in the vessel to a temperature equal to or more than 60° C.

7. The process of claim 6, wherein the mixture to be stripped is heated before being fed into the vessel and/or heated in the vessel to a temperature equal to or higher than 75° C.

8. The process according to claim 1, wherein said stripping is performed continuously.

9. The process according to claim 8, wherein said stripping is performed continuously in a stripping column.

10. The process according to claim 9, wherein the mixture to be stripped is heated to a temperature equal to or higher than 80° C. before it is introduced into the stripping column.

11. The process according to claim 5, wherein the inert gas is heated to a temperature between 100° C. and 150° C.

12. The process according to claim 8, wherein the inert gas is heated to a temperature between 100° C. and 150° C.

13. The process according to claim 1, wherein nitrogen is applied as the inert gas.

14. The process according to claim 1, wherein the hydrogen fluoride content is reduced by said stripping to be equal to or less than 2% by weight of the mixture.

15. The process according to claim 14, where the hydrogen fluoride content is reduced to be equal to or less than 0.1% by weight.

16. A process for the manufacture of purified organic carbonate from its mixture with HF, comprising stripping HF from said mixture containing said organic carbonate and HF by passing an inert gas through said mixture to obtain a product depleted in HF, and further comprising at least one subsequent step of distillation of the product depleted in HF.

17. The process according to claim 16, wherein the mixture which is stripped is a mixture comprising HF and a fluorosubstituted alkylene carbonate.

18. The process according to claim 1, wherein the mixture which is stripped is a mixture comprising a fluorosubstituted alkylene carbonate and hydrogen fluoride.

19. The process according to claim 1, wherein the mixture which is stripped is a mixture comprising a fluorosubstituted dialkyl carbonate and hydrogen fluoride.
